(19) United States
(12) Patent Application Publication
(10) Pub. No.: US 2022/0002297 A1
(43) Pub. Date: Jan. 6, 2022

(54) POLYMORPHS OF X842

(71) Applicant: Cinclus Pharma AG, Basel (CH)

(72) Inventors: Mikael DAHLSTRÖM, Mölndal (SE); Pingsheng HU, Guiyang (CN); Davide SBARBADA, Cura Carpignano (IT); Matteo ZACCHÈ, Nerviano (IT); Marinus BOUMA, Uppsala (SE)

(21) Appl. No.: 17/365,474

(22) Filed: Jul. 1, 2021

Related U.S. Application Data
(60) Provisional application No. 63/047,607, filed on Jul. 2, 2020.

(51) Int. Cl. C07D 471/04

(52) U.S. Cl. CPC C07D 471/04; C07B 2200/13

(57) ABSTRACT

The present invention relates to polymorphs of 5-{2-[({8-[(2,6-dimethylbenzyl)amino]-2,3-dimethylimidazo[1,2-a]pyridine-6-yl}carbonyl)-amino]ethoxy}-5-oxopentanoic acid (X842), more specifically forms A and B of X842. The invention also relates to a process for the preparation of these polymorphs, to pharmaceutical compositions comprising such polymorphs, and to methods for treating or preventing a gastrointestinal inflammatory disease or a gastric acid related disease, comprising administering a pharmaceutical composition comprising such polymorphs.

### CROSS REFERENCE TO RELATED APPLICATIONS

This application claims the benefit of priority to U.S. Provisional Application No. 63/047,607 filed on Jul. 2, 2020, the contents of which is hereby incorporated by reference in its entirety.

### TECHNICAL FIELD

The present invention relates to polymorphs of 5-{2-[({8-[(2,6-dimethylbenzyl)amino]-2,3-dimethylimidazo[1,2-a]pyridine-6-yl}carbonyl)-amino]ethoxy}-5-oxopentanoic acid (X842), more specifically forms A and B of X842. The invention also relates to a process for the preparation of these polymorphs, to pharmaceutical compositions comprising such polymorphs, and to methods for treating or preventing a gastrointestinal inflammatory disease or a gastric acid related disease, comprising administering a pharmaceutical composition comprising such polymorphs.

### BACKGROUND

The compound 5-{2-[({8-[(2,6-dimethylbenzyl)amino]-2,3-dimethylimidazo[1,2-a]pyridine-6-yl}carbonyl)-amino]ethoxy}-5-oxopentanoic acid (X842; structure shown below) is disclosed in WO 2010/063876. It is a potassium-competitive acid blocker (P-CAB), which competitively inhibits the gastric hydrogen potassium pump (H⁺/K⁺ ATPase) in the parietal cells. X842 may therefore be used to control the secretion of gastric acid in the stomach.

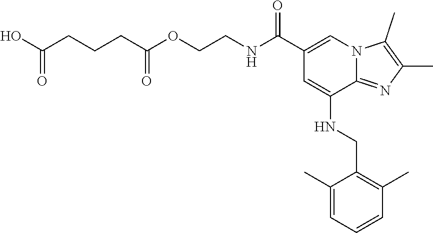 [SMILES: Cc1cccc(C)c1CNc1cc(C(=O)NCCOC(=O)CCCC(=O)O)cn2c(C)c(C)nc12]

X842 is a prodrug of Linaprazan, which was disclosed in WO 99/55706 and previously studied in Phase I and II studies. These studies showed that Linaprazan was well tolerated, with a fast onset of action and full effect at first dose. However, Linaprazan was quickly eliminated from the body and had too short duration of acid inhibition. In comparison, X842 has a longer half-life in the body and shows total control of the gastric acid production for a longer time compared to Linaprazan. A clinical Phase I study has shown that administration of a single dose of X842 can maintain the intragastric acidity above pH 4 for 24 hours. X842 is therefore tailored for patients with severe erosive gastroesophageal reflux disease (eGERD).

For use in pharmaceutical preparations, it is desirable that the active pharmaceutical ingredient (API) is in a highly crystalline form. Non-crystalline (i.e., amorphous) materials may contain higher levels of residual solvents, which is undesirable. Also, because of their lower chemical and physical stability, as compared with crystalline material, amorphous materials may display faster decomposition and may spontaneously form crystals with a variable degree of crystallinity. This may result in unreproducible solubility rates and difficulties in storing and handling the material. Thus, there is a need for crystalline forms of X842 having improved properties with respect to stability, bulk handling and solubility. In particular, it is an object of the present invention to provide a stable crystalline form of X842 that contains low levels of residual solvents, has a high chemical stability and low hygroscopicity and can be obtained in high levels of crystallinity.

### BRIEF DESCRIPTION OF THE DRAWINGS

FIG. 1 shows the X-ray powder diffractogram of Form A.

FIG. 2 shows the X-ray powder diffractogram of Form B.

FIG. 3 shows the differential scanning calorimetry (DSC) thermogram of Form A.

FIG. 4 shows the DSC thermogram of Form B.

FIG. 5 shows an overlay of the DSC thermograms of Forms A and B.

FIG. 6 shows the thermogravimetric analysis (TGA) and the heatflow thermograms of Form A.

FIG. 7 shows the TGA and the heatflow thermograms of Form B.

FIG. 8 shows an overlay of the TGA and heatflow thermograms of Forms A and B.

FIG. 9 shows the dynamic vapour sorption (DVS) isotherm plot of Form A.

FIG. 10 shows the X-ray powder diffractogram of Form A before (top) and after (bottom) DVS.

### DETAILED DESCRIPTION OF THE INVENTION

It has been discovered that under certain conditions, stable crystalline forms (polymorphs) of X842 can be obtained. Investigation of these crystalline forms have shown that they are anhydrates. The forms show good stability and are therefore useful in pharmaceutical compositions of X842. In a first aspect, therefore, the invention relates to a crystalline anhydrate of X842.

Some crystalline anhydrates of X842 were found to have a low hygroscopicity, with a water uptake of only about 0.1% at 25° C./80% RH. This low hygroscopicity is considered advantageous, as the water content of the crystals remains substantially constant even with humidity changes within the normal relative humidity range of about 30% to about 70% RH. In some embodiments, the invention provides a crystalline anhydrate of X842, where the anhydrate is stable at a relative humidity (RH) up to 80% at a temperature of 25° C. In some embodiments, the invention provides a crystalline anhydrate of X842, where the anhydrate is stable at a relative humidity (RH) up to 60% at a temperature of 25° C. Such anhydrates can be stable under these conditions for at least 1 day, 1 week, 1 month, 3 months, 6 months, 1 year, 2 years, 3 years or even longer.

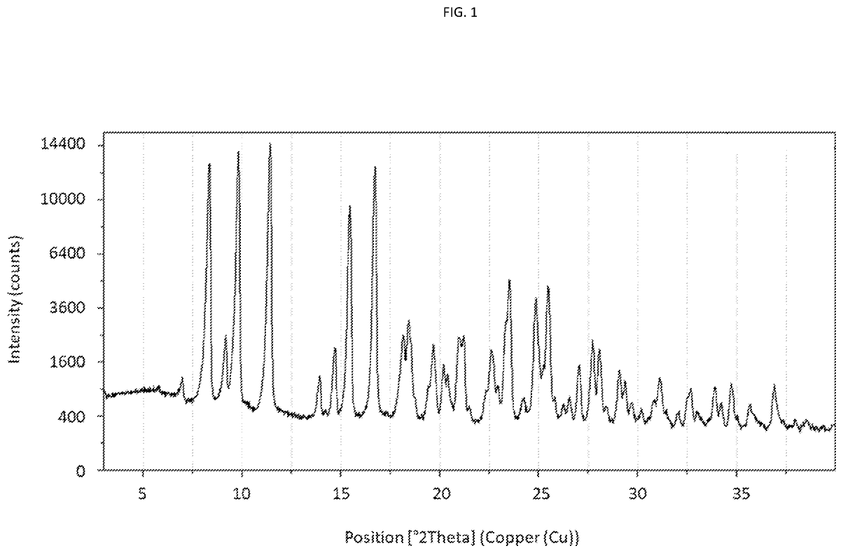

In one embodiment, the crystalline anhydrate is Form A. Form A may be prepared by anti-solvent or reverse anti-solvent addition from certain solvent combinations. In one embodiment, Form A has an X-ray powder diffraction (XRPD) pattern, obtained with CuKα1-radiation, with at least peaks at °2θ values of 9.9±0.2 and 11.5±0.2. In some embodiments, Form A has an XRPD pattern, obtained with CuKα1-radiation, with at least peaks at °2θ values of 9.9±0.2 and 11.5±0.2 and one or more of 8.4±0.2, 15.5±0.2 and 16.8±0.2. In some embodiments, Form A has an XRPD pattern, obtained with CuKα1-radiation, with at least peaks at °2θ values of 8.4±0.2, 9.9±0.2, 11.5±0.2, 15.5±0.2 and 16.8±0.2. In some embodiments, Form A has an XRPD pattern, obtained with CuKα1-radiation, with at least peaks at °2θ values of 8.4±0.2, 9.9±0.2, 11.5±0.2, 15.5±0.2, 16.8±0.2, 23.5±0.2, 24.9±0.2 and 25.5±0.2. In some embodiments, Form A has an XRPD pattern, obtained with CuKα1-radiation, with at least peaks at °2θ values of 8.4±0.2, 9.9±0.2, 11.5±0.2, 15.5±0.2, 16.8±0.2, 23.5±0.2, 24.9±0.2 and 25.5±0.2, and one or more of 18.2±0.2, 18.4±0.2, 21.0±0.2, 21.2±0.2 and 23.3±0.2. In some embodiments, Form A has an XRPD pattern, obtained with CuKα1-radiation, with at least peaks at °2θ values of 8.4±0.2, 9.9±0.2, 11.5±0.2, 15.5±0.2, 16.8±0.2, 18.2±0.2, 18.4±0.2, 21.0±0.2, 21.2±0.2, 23.3±0.2, 23.5±0.2, 24.9±0.2 and 25.5±0.2. In a particular embodiment, the invention relates to Form A, having an XRPD pattern, obtained with CuKα1-radiation, substantially as shown in FIG. 1.

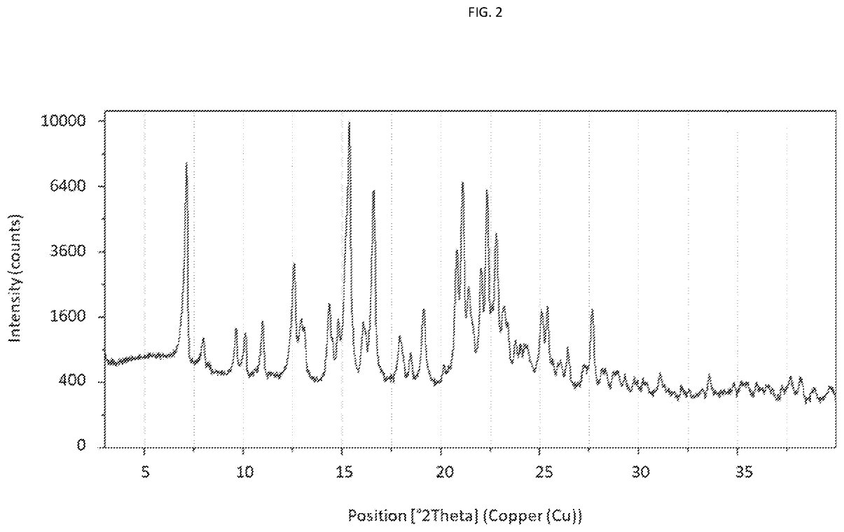

In another embodiment, the crystalline anhydrate is Form B. This form may be prepared by reverse anti-solvent addition from certain solvent combinations. In one embodiment, Form B has an XRPD pattern, obtained with CuKα1-radiation, with at least peaks at °2θ values of 7.2±0.2 and 15.4±0.2. In some embodiments, Form B has an XRPD pattern, obtained with CuKα1-radiation, with at least peaks at °2θ values of 7.2±0.2 and 15.4±0.2. and one or more of 16.6±0.2, 21.1±0.2 and 22.3±0.2. In some embodiments, Form B has an XRPD pattern, obtained with CuKα1-radiation, with at least peaks at °2θ values of 7.2±0.2, 15.4±0.2, 16.6±0.2, 21.1±0.2 and 22.3±0.2. In some embodiments, Form B has an XRPD pattern, obtained with CuKα1-radiation, with at least peaks at °2θ values of 7.2±0.2, 12.6±0.2, 15.4±0.2, 16.6±0.2, 20.8±0.2, 21.1±0.2, 22.3±0.2 and 22.8±0.2. In some embodiments, Form B has an XRPD pattern, obtained with CuKα1-radiation, with at least peaks at °2θ values of 7.2±0.2, 12.6±0.2, 15.4±0.2, 16.6±0.2, 20.8±0.2, 21.1±0.2, 22.3±0.2 and 22.8±0.2, and one or more of 14.4±0.2, 19.1±0.2, 21.4±0.2, 22.0±0.2 and 27.7±0.2. In some embodiments, Form B has an XRPD pattern, obtained with CuKα1-radiation, with at least peaks at °2θ values of 7.2±0.2, 12.6±0.2, 14.4±0.2, 15.4±0.2, 16.6±0.2, 19.1±0.2, 20.8±0.2, 21.1±0.2, 21.4±0.2, 22.0±0.2, 22.3±0.2 and 22.8±0.2 and 27.7±0.2. In a particular embodiment, the invention relates to Form B, having an XRPD pattern, obtained with CuKα1-radiation, substantially as shown in FIG. 2.

In another aspect, the invention relates to a composition comprising a crystalline anhydrate of X842. In some embodiments, the composition comprises Form A. In some embodiments, the composition comprising Form A can be substantially pure. In some embodiments, the composition comprising Form A has a polymorphic purity of at least about 90%. In some embodiments, the composition has a polymorphic purity of at least about 95%. In some embodiments, the composition has a polymorphic purity of at least about 98%. For example, the composition can have a polymorphic purity of at least about 98.5%, such as at least about 99%, such as at least about 99.5%, such as at least about 99.8%, or such as at least about 99.9%. In some embodiments, the composition comprising Form A is substantially free of other forms of X842. For example, in some embodiments, the composition is substantially free of other anhydrous forms of X842, such as Form B of X842. In some embodiments, the composition comprising Form A contains less than about 15% by weight of Form B of X842 or any other polymorphic form. For example, the composition contains less than about 14%, about 13%, about 12%, about 11%, about 10%, about 9%, about 8%, about 7%, about 6%, about 5%, about 4%, about 3%, about 2%, about 1% or less by weight of form B of X842 or any other polymorphic form.

In some embodiments, the composition comprises Form B. In some embodiments, the composition comprising Form B can be substantially pure. In some embodiments, the composition comprising Form B has a polymorphic purity of at least about 90%. In some embodiments, the composition has a polymorphic purity of at least about 95%. In some embodiments, the composition has a polymorphic purity of at least about 98%. For example, the composition can have a polymorphic purity of at least about 98.5%, such as at least about 99%, such as at least about 99.5%, such as at least about 99.8%, or such as at least about 99.9%. In some embodiments, the composition comprising Form B is substantially free of other forms of X842. For example, in some embodiments, the composition is substantially free of other anhydrous forms of X842, such as Form A of X842. In some embodiments, the composition comprising Form B contains less than about 15% by weight of Form A or any other polymorph of X842. For example, the composition contains less than about 14%, about 13%, about 12%, about 11%, about 10%, about 9%, about 8%, about 7%, about 6%, about 5%, about 4%, about 3%, about 2%, about 1% or less by weight of form A or any other polymorph of X842.

In another aspect, the invention relates to a pharmaceutical composition comprising a therapeutically effective amount of a crystalline anhydrate of X842 as described herein, in association with one or more pharmaceutically acceptable excipients. The excipients may e.g. include surfactants, fillers, binders, disintegrants, glidants and lubricants. In some embodiments, the anhydrate is Form A. In some embodiments, the anhydrate is Form B.

In some embodiments, the pharmaceutical composition comprises form A having a polymorphic purity of at least about 90%. In some embodiments, the polymorphic purity is at least about 95%. In some embodiments, the polymorphic purity is at least about 98%. For example, the polymorphic purity is at least about 98.5%, such as at least about 99%, such as at least about 99.5%, such as at least about 99.8%, or such as at least about 99.9%. In some embodiments, the pharmaceutical composition comprising Form A is substantially free of other forms of X842. For example, in some embodiments, the pharmaceutical composition is substantially free of other anhydrous forms of X842, such as Form B of X842. In some embodiments, Form A contains less than about 15% by weight of Form B or any other polymorph of X842. For example, Form A contains less than about 14%, about 13%, about 12%, about 11%, about 10%, about 9%, about 8%, about 7%, about 6%, about 5%, about 4%, about 3%, about 2%, about 1% or less by weight of form B or any other polymorph of X842.

In some embodiments, the pharmaceutical composition can comprise between about 1% and about 100%, such as between about 1% and about 50%, or such as between about 1% and about 20% by weight of a crystalline anhydrate of X842. For example, the composition can comprise between about 1% and about 15%, or between about 5% and about 20%, such as between about 1% and about 10%, between about 5% and about 15%, and between about 10% and about 20%, or such as between about 1% and about 5%, between about 5% and about 10%, between about 10% and about 15%, and between about 15% and about 20% by weight of a crystalline anhydrate of X842. In some embodiments, the composition comprises about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2% or about 1% by weight of a crystalline anhydrate of X842.

In some embodiments, the composition comprises a unit dose of about 25 mg to about 150 mg of a crystalline anhydrate of X842. For example, the composition can comprise between about 25 mg and about 50 mg, between about 50 mg and about 75 mg, between about 75 mg and about 100 mg, between about 100 mg and about 125 mg, or between about 125 mg and about 150 mg. In some embodiments, the composition comprises about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg of a crystalline anhydrate of X842. The daily dose can be administered as a single dose or divided into two, three or more unit doses.

Following absorption into the blood stream, X842 is quickly metabolized into Linaprazan, which is the active metabolite of X842. Whereas the plasma concentration of X842 is only very low and difficult to determine, the plasma concentration of Linaprazan may be determined instead. Phase I studies have indicated that certain doses of X842 should be able to maintain the intra-gastric pH above 4 for 24 hours after administration. It is estimated that this requires a minimal plasma concentration (Cₘᵢₙ) of Linaprazan of at least about 240 nmol/L after 22 hours. At such doses, a once daily oral administration of the formulation would be sufficient. Therefore, in another aspect, the invention relates to the pharmaceutical composition as disclosed herein, wherein a unit dose of the composition provides a Cₘᵢₙ of Linaprazan in a human of at least about 240 nmol/L after 22 hours following oral administration of the pharmaceutical composition to said human.

In some embodiments, the pharmaceutical composition comprises a surfactant. The surfactant may be a cationic surfactant, an anionic surfactant or a nonionic surfactant. Examples of cationic surfactants include, but are not limited to, cetyltrimethylammonium bromide (cetrimonium bromide) and cetylpyridinium chloride. Examples of anionic surfactants include, but are not limited to, sodium dodecyl sulfate (sodium lauryl sulfate) and ammonium dodecyl sulfate (ammonium lauryl sulfate). Examples of nonionic surfactants include, but are not limited to, glycerol monooleate, glycerol monostearate, polyoxyl castor oil (Cremophor EL), poloxamers (e.g., poloxamer 407 or 188), polysorbate 80 and sorbitan esters (Tween). In some embodiments, the surfactant is an anionic surfactant.

In some embodiments, the pharmaceutical composition comprises a filler. Examples of suitable fillers include, but are not limited to, dicalcium phosphate dihydrate, calcium sulfate, lactose (such as lactose monohydrate), sucrose, mannitol, sorbitol, cellulose, microcrystalline cellulose, dry starch, hydrolyzed starches and pregelatinized starch.

In some embodiments, the pharmaceutical composition comprises a binder. Examples of suitable binders include, but are not limited to, starch, pregelatinized starch, gelatin, sugars (such as sucrose, glucose, dextrose, lactose and sorbitol), polyethylene glycol, waxes, natural and synthetic gums (such as acacia gum and tragacanth gum), sodium alginate, cellulose derivatives (such as hydroxypropylmethylcellulose (or hypromellose), hydroxypropylcellulose and ethylcellulose) and synthetic polymers (such as acrylic acid and methacrylic acid copolymers, methacrylic acid copolymers, methyl methacrylate copolymers, aminoalkyl methacrylate copolymers, polyacrylic acid/polymethacrylic acid copolymers and polyvinylpyrrolidone (povidone)).

In some embodiments, the pharmaceutical composition comprises a disintegrant. Examples of suitable disintegrants include, but are not limited to, dry starch, modified starch (such as (partially) pregelatinized starch, sodium starch glycolate and sodium carboxymethyl starch), alginic acid, cellulose derivatives (such as sodium carboxymethylcellulose, hydroxypropyl cellulose, and low substituted hydroxypropyl cellulose (L-HPC)) and cross-linked polymers (such as carmellose, croscarmellose sodium, carmellose calcium and cross-linked PVP (crospovidone)).

In some embodiments, the pharmaceutical composition comprises a glidant or lubricant. Examples of suitable glidants and lubricants include, but are not limited to, talc, magnesium stearate, calcium stearate, sodium stearyl fumarate, stearic acid, glyceryl behenate, colloidal anhydrous silica, aqueous silicon dioxide, synthetic magnesium silicate, fine granulated silicon oxide, starch, sodium lauryl sulfate, boric acid, magnesium oxide, waxes (such as carnauba wax), hydrogenated oil, polyethylene glycol, sodium benzoate, polyethylene glycol, and mineral oil.

In general, pharmaceutical compositions may be prepared in a conventional manner using conventional excipients. In some embodiments, the ingredients of the formulation are mixed to a homogenous mixture and then formulated as tablets or capsules. The homogenous mixture of the ingredients may be compressed into tablets using conventional techniques, such as rotary tablet press. Alternatively, the mixture may be wetted by the addition of a liquid, such as water and/or an appropriate organic solvent (e.g., ethanol or isopropanol), and thereafter granulated and dried. The granules obtained may be then be compressed into tablets using conventional techniques. Capsules may comprise a powder mixture or small multiparticulates (such as granules, extruded pellets or minitablets) of the ingredients. In some embodiments, the formulation is in the form of a tablet.

The crystalline anhydrates of X842 disclosed herein can be used in the treatment or prevention of diseases or conditions wherein inhibition of gastric acid secretion is necessary or desirable, such as in H. pylori eradication. Examples of such diseases and conditions include gastrointestinal inflammatory diseases and gastric acid related diseases, such as gastritis, gastroesophageal reflux disease (GERD), erosive gastroesophageal reflux disease (eGERD), H. pylori infection, Zollinger-Ellison syndrome, peptic ulcer disease (including gastric ulcers and duodenal ulcers), bleeding gastric ulcer, symptoms of gastroesophageal reflux disease (including heartburn, regurgitation and nausea), gastrinoma and acute upper gastrointestinal bleeding.

In one aspect, therefore, the invention relates to a method for treating or preventing a gastrointestinal inflammatory disease or a gastric acid related disease in a subject in need thereof, comprising administering a pharmaceutical composition comprising a therapeutically effective amount of a crystalline anhydrate of X842, as disclosed herein. In some embodiments, the crystalline anhydrate of X842 is Form A. In some embodiments, the crystalline anhydrate of X842 is Form B.

In some embodiments, the crystalline anhydrate of X842 is substantially present as Form A, having an X-ray powder diffraction (XRPD) pattern, obtained with CuKα1-radiation, with at least peaks at °2θ values of 9.9±0.2 and 11.5±0.2. In some embodiments, Form A is substantially free of other anhydrous forms of X842, such as Form B of X842. In some embodiments, Form A contains less than about 15% by weight of Form B of X842, such as less than about 10%, about 9%, about 8%, about 7%, about 6%, about 5, about 4%, about 3%, about 2% or such as less than about 1% by weight of Form B of X842.

In some embodiments, the treatment of GERD is on-demand treatment of GERD.

In another aspect, the invention relates to a pharmaceutical composition comprising a therapeutically effective amount of a crystalline anhydrate of X842, as disclosed herein, for use in the treatment or prevention of a gastrointestinal inflammatory disease or a gastric acid related disease.

In another aspect, the invention relates to a process for the preparation of Forms A and B of X842. In some embodiments, Forms A and B can be formed by anti-solvent addition or reverse anti-solvent addition. In some embodiments, the solvent is NMP, DMA, DMF or DMSO. In some embodiments, the anti-solvent is acetonitrile, acetone, ethyl acetate, isopropyl acetate, methyl tert-butyl ether, n-heptane, water, methanol, ethanol, methyl isobutyl ketone, toluene or dichloromethane.

In some embodiments, Form A is formed by anti-solvent addition comprising NMP as the solvent and acetonitrile, acetone, ethyl acetate or methyl tert-butyl ether as the anti-solvent. In some embodiments, Form A is formed by anti-solvent addition comprising DMA as the solvent and isopropyl acetate or n-heptane as the anti-solvent. In some embodiments, Form A is formed by reverse anti-solvent addition comprising NMP as the solvent and isopropyl acetate or n-heptane as the anti-solvent. In some embodiments, Form A is formed by reverse anti-solvent addition comprising DMA as the solvent and ethanol, methyl isobutyl ketone or toluene as the anti-solvent.

In some embodiments, Form B is formed by reverse anti-solvent addition comprising NMP as the solvent and water as the anti-solvent. In some embodiments, Form B is formed by reverse anti-solvent addition comprising DMSO as the solvent and methanol as the anti-solvent. In some embodiments, Form B is formed by reverse anti-solvent addition comprising DMA as the solvent and water as the anti-solvent. In some embodiments, Form B is formed by reverse anti-solvent addition comprising DMF as the solvent and dichloromethane as the anti-solvent.

In some embodiments, the process comprises the steps of:

a) preparing a solution of X842 in a suitable solvent;

b) optionally adding a suitable anti-solvent to the solution of step a);

c) maintaining stirring until a solid is obtained;

d) recovering the solid obtained in step c); and

e) drying the solid under vacuum.

It has been observed that in some cases, the crystallization of the desired anhydrous form of X842 does not lead to pure Form A or Form B but to a mixture of Forms A and B, e.g. a composition primarily comprising Form A but also about 1%, 2%, 3%, 4%, 5%, 6%, 7%, 8%, 9%, 10%, 11%, 12%, 13%, 14%, 15% or more by weight of Form B. In order to improve the polymorphic purity of the composition, seed crystals of the desired form can be added to the solution from which the anhydrate should crystallize.

In some embodiments, therefore, the process comprises the steps of:

a) preparing a solution of X842 in a suitable solvent;

b) optionally adding a suitable anti-solvent to the solution of step a);

c) adding seed crystals to the solution of step b);

d) maintaining stirring until a solid is obtained;

e) recovering the solid obtained in step d); and

f) drying the solid under vacuum.

The polymorphic purity of crystalline Form A can additionally be improved by maintaining stirring (or slurrying) of the obtained solid in a suitable solvent or solvent mixture for an extended period of time, such as at least about 1 hour, about 2 hours, about 3 hours, about 4 hours, about 6 hours, about 8 hours, about 10 hours, about 12 hours, about 16 hours, about 20 hours or such as at least about 24 hours. In some embodiments, slurrying is performed in the solvent or solvent mixture from which the anhydrate crystallized. In some embodiments, the crystals are collected after crystallization, optionally dried, and then slurried in another solvent or solvent mixture. In some embodiments, slurrying is performed in methanol, ethanol, isopropyl alcohol, acetonitrile, acetone, ethyl acetate, tetrahydrofuran, 1,4-dioxane, toluene, n-heptane, dichloromethane, chloroform, water, methyl isobutyl ketone, isopropyl acetate, methyl tert-butyl ether, NMP, DMA, DMF or DMSO, or in a mixture of two of these solvents. In some embodiments, slurrying is performed in methanol. In some embodiments, slurrying is performed in a mixture of methanol and dichloromethane. In some embodiments, slurrying is performed in DMF. In some embodiments, slurrying is performed in a mixture of DMF and acetone.

In some embodiments, slurrying is performed at room temperature. In some embodiments, slurrying is performed at elevated temperatures, such as at about 35° C., about 40° C., about 45° C., about 50° C., about 55° C., about 60° C., about 65° C., about 70° C., about 75° C., about 80° C., about 85° C., about 90° C., about 95° C., or such as at about 100° C.

In some embodiments, therefore, the process comprises the steps of:

a) preparing a solution of X842 in a suitable solvent;

b) optionally adding a suitable anti-solvent to the solution of step a);

c) adding seed crystals to the solution of step b);

d) maintaining stirring until a solid is obtained;

e) maintaining stirring of the slurry of step d) for an extended period of time;

f) recovering the solid obtained in step e); and

g) drying the solid under vacuum.

In other embodiments, the process comprises the steps of:

a) preparing a solution of X842 in a suitable solvent;

b) optionally adding a suitable anti-solvent to the solution of step a);

c) adding seed crystals to the solution of step b);

d) maintaining stirring until a solid is obtained;

e) recovering the solid obtained in step d);

f) optionally drying the solid;

g) preparing a slurry of the solid of step f) in a suitable solvent or solvent mixture;

h) maintaining stirring of the slurry of step g) for an extended period of time;

i) recovering the solid obtained in step h); and

j) drying the solid under vacuum.

As used herein, the term “polymorph” refers to crystals of the same molecule that have different physical properties as a result of the order of the molecules in the crystal lattice. Polymorphs of a single compound have one or more different chemical, physical, mechanical, electrical, thermodynamic, and/or biological properties from each other. Differences in physical properties exhibited by polymorphs can affect pharmaceutical parameters such as storage stability, compressibility, density (important in composition and product manufacturing), dissolution rates (an important factor in determining bioavailability), solubility, melting point, chemical stability, physical stability, powder flowability, water sorption, compaction, and particle morphology. Differences in stability can result from changes in chemical reactivity (e.g. differential oxidation, such that a dosage form discolours more rapidly when comprised of one polymorph than when comprised of another polymorph) or mechanical changes (e.g., crystal changes on storage as a kinetically favoured polymorph converts to a thermodynamically more stable polymorph) or both (e.g., one polymorph is more hygroscopic than the other). As a result of solubility/dissolution differences, some transitions affect potency and/or toxicity. In addition, the physical properties of the crystal may be important in processing; for example, one polymorph might be more likely to form solvates or might be difficult to filter and wash free of impurities (i.e., particle shape and size distribution might be different between one polymorph relative to the other). “Polymorph” does not include amorphous forms of the compound.

As used herein, the term “amorphous” refers to a non-crystalline form of a compound which may be a solid state form of the compound or a solubilized form of the compound. For example, “amorphous” refers to a compound without a regularly repeating arrangement of molecules or external face planes.

As used herein, the term “anhydrate” or “anhydrous form” refers to a polymorph of X842 that has 1% or less by weight water, for example 0.5% or less, 0.25% or less, or 0.1% or less by weight water.

As used herein, the term “polymorphic purity” when used in reference to a composition comprising a polymorph of X842, refers to the percentage of one specific polymorph relative to another polymorph or an amorphous form of X842 in the referenced composition. For example, a composition comprising Form A having a polymorphic purity of 90% would comprise 90 weight parts Form A and 10 weight parts of other crystalline and/or amorphous forms of X842.

As used herein, the terms “effective amount” or “therapeutically effective amount” refer to a sufficient amount of X842 that, following administration to a subject, will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic use is the amount of X842 required to provide a clinically significant decrease in disease symptoms. An appropriate “effective” amount in any individual case is determined using any suitable technique, such as a dose escalation study.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “pharmaceutically acceptable” refers to those compounds, materials, compositions and/or dosage forms that are suitable for human pharmaceutical use and that are generally safe, non-toxic and neither biologically nor otherwise undesirable.

As used herein, a compound or composition is “substantially free” of one or more other components if the compound or composition contains no significant amount of such other components. Such components can include starting materials, residual solvents, or any other impurities that can result from the preparation of and/or isolation of the compounds and compositions provided herein. In some embodiments, a polymorph form provided herein is substantially free of other polymorph forms. In some embodiments, a particular polymorph of X842 is “substantially free” of other polymorphs if the particular polymorph constitutes at least about 95% by weight of X842 present. In some embodiments, a particular polymorph of X842 is “substantially free” of other polymorphs if the particular polymorph constitutes at least about 97%, about 98%, about 99%, or about 99.5% by weight of X842 present.

As used herein, a compound is “substantially present” as a given polymorph if at least about 50% by weight of the compound is in the form of that polymorph, for example if at least about 60%, at least about 70%, at least about 80%, or at least about 90% by weight of the compound is in the form of that polymorph. In some embodiments, at least about 95%, such as at least about 96%, such as at least about 97%, such as at least about 98%, such as at least about 99% or such as at least about 99.5% by weight of the compound is in the form of that polymorph.

As used herein, the term “stable” means that the polymorphs do not exhibit a change in one or more of polymorph form (e.g., an increase or decrease of a certain form), appearance, pH, percent impurities, activity (as measured by in vitro assays), or osmolarity over time. In some embodiments, the polymorphs provided herein are stable for at least 1, 2, 3 or 4 weeks. For example, the polymorphs do not exhibit a change in one or more of polymorph form (e.g., an increase or decrease of a certain form), appearance, pH, percent impurities, activity (as measured by in vitro assays), or osmolarity over at least 1, 2, 3 or 4 weeks. In some embodiments, the polymorphs provided herein are stable for at least 1, 2, 3, 4, 5, 6, 7, 8, 9, 10, 11 or 12 months. For example, the polymorphs do not exhibit a change in one or more of polymorph form (e.g., an increase or decrease of a certain form), appearance, pH, percent impurities, activity (as measured by in vitro assays), or osmolarity over at least 1, 2, 3, 4, 5, 6, 7, 8, 9, 10, 11 or 12 months. In the above, the phrase “do not exhibit a change” refers to a change of less than 5% (e.g., less than 4%, less than 3%, less than 2%, less than 1%) as measured for any of the parameters over the relevant time period.

The crystallinity of a polymorph of X842 may be measured e.g. by X-Ray Powder Diffraction (XRPD) methods or by Differential Scanning calorimetry (DSC) methods. When reference is made herein to a crystalline compound, preferably the crystallinity is greater than about 70%, such as greater than about 80%, particularly greater than about 90%, more particularly greater than about 95%. In some embodiments, the degree of crystallinity is greater than about 98%. In some embodiments, the degree of crystallinity is greater than about 99%. The % crystallinity refers to the percentage by weight of the total sample mass which is crystalline.

As used herein, the term “about” refers to a value or parameter herein that includes (and describes) embodiments that are directed to that value or parameter per se. For example, description referring to “about 20” includes description of “20.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater.

The invention will now be described by the following examples which do not limit the invention in any respect. All cited documents and references mentioned herein are incorporated by reference in their entireties.

### Abbreviations

DCM dichloromethane

DMA N,N-dimethylacetamide

DMF N,N-dimethylformamide

DMSO dimethyl sulfoxide

EtOAc ethyl acetate

EtOH ethanol

MeCN acetonitrile

MeOH methanol

MIBK methyl isobutyl ketone

MTBE methyl tert-butyl ether

NMP N-methyl-2-pyrrolidone

RH relative humidity

Experimental Methods

X-Ray Powder Diffraction (XRPD) Analysis

Analyses were performed on a PANalytical X'Pert Pro diffractometer equipped with a Cu long fine focus X-ray tube and an XCelerator detector. Fixed divergence and anti-scatter slits were used together with 0.04 rad Soller slits and a Ni-filter. Samples were analysed between 3-40° in 2-theta, with a step size of 0.017. The following experimental settings were used:
Tube tension and current: 40 kV, 40 mA
Wavelength alpha1 (CuKα1): 1.5406 Å
Wavelength alpha2 (CuKα2): 1.5444 Å
Wavelength alpha1 and alpha2 mean (CuKα): 1.5419 Å

It is known in the art that an X-ray powder diffraction pattern may be obtained having one or more measurement errors depending on measurement conditions (such as equipment, sample preparation or machine used). In particular, it is generally known that intensities in an XRPD pattern may fluctuate depending on measurement conditions and sample preparation. For example, persons skilled in the art of XRPD will realize that the relative intensities of peaks may vary according to the orientation of the sample under the test and on the type and setting of the instrument used. The skilled person will also realize that the position of reflections can be affected by the precise height at which the sample sits in the diffractometer and the zero calibration of the diffractometer. The surface planarity of the sample may also have a small effect. Hence a person skilled in the art will appreciate that the diffraction pattern presented herein is not to be construed as absolute and any crystalline form that provides a powder diffraction pattern substantially identical to those disclosed herein fall within the scope of the present disclosure (for further information, see R. Jenkins and R. L. Snyder, “Introduction to X-ray powder diffractometry”, John Wiley & Sons, 1996).

Thermogravimetric Analysis (TGA)

Analyses were performed on a Mettler Toledo TGA/DSC1 instrument, coupled with a Thermo Nicolet iS10 spectrometer. The sample was flushed with dry nitrogen gas during the analysis. The sample was scanned from 25 to 320° C. with a scan rate of 10° C./minute.

Differential Scanning Calorimetry (DSC)

Analyses were performed on a Mettler Toledo DSC1 instrument. The sample was flushed with dry nitrogen gas during the analysis. The sample was scanned from 25 to 260° C. with a scan rate of 10° C./minute.

Dynamic Vapour Sorption (DVS)

Analyses were performed on a Surface Measurement Systems DVS Intrinsic instrument. The relative humidity at 25° C. was calibrated against the deliquescense point of LiCL, Mg(NO₃)₂ and KCl. The sample (about 10-20 mg) was dried with nitrogen gas until a dm/dt below 0.002% per minute was reached. The instrument was running in dm/dt mode using a minimum dm/dt stability duration of 10 minutes and a maximum equilibrium time of 180 minutes. The sample was subjected to a sorption-desorption cycle running from 0 to 95 to 0% RH. One cycle consisted of 20 steps, those between 0 and 90% RH were taken in 10% RH each.

### EXAMPLES

### Example 1

Preparation of Form A

Anti-Solvent Addition:

About 15 mg of sample material was dissolved in 0.3-0.55 mL solvent to obtain a clear solution. The solution was magnetically stirred, followed by addition of relative anti-solvent to induce crystallization, or until the total amount of anti-solvent reached 2 mL. The crystals were isolated and analyzed with XRPD. The solvent pairs that gave rise to Form A are listed in Table 1 below.

TABLE 1
Summary of anti-solvent addition
experiments that generated Form A

| Solvent | Anti-solvent | Solid Form |
|---|---|---|
| NMP | MeCN | Form A |
| NMP | Acetone | Form A |
| NMP | EtOAc | Form A |
| NMP | MTBE | Form A |
| DMA | Isopropyl acetate | Form A* |
| DMA | n-Heptane | Form A** |

*solution was cooled to −20° C. to induce crystallization
**solution at room temperature was evaporated to induce crystallization

Reverse Anti-Solvent Addition:

About 15 mg of sample material was dissolved in appropriate solvent to obtain a saturated solution. The saturated solution was added into a 20-mL glass vial with 5 mL of relative anti-solvent and the mixture was stirred at room temperature to induce crystallization. The crystals were agitated for 5 minutes and isolated and then analyzed with XRPD. The solvent pairs that gave rise to Form B are listed in Table 2 below.

TABLE 2
Summary of reverse anti-solvent addition
experiments that generated Form A

| Solvent | Anti-solvent | Solid Form |
|---|---|---|
| NMP | Isopropyl acetate | Form A |
| NMP | n-Heptane | Form A |
| DMA | EtOH | Form A |
| DMA | MIBK | Form A |
| DMA | Toluene | Form A |

The XRPD peaks for Form A are listed in Table 3 below. The diffractogram for Form A is shown in FIG. 1.

TABLE 3
XRPD peaks of Form A

| Position | Heights | FWHM Left | d-spacing | Rel. Int. | Area |
|---|---|---|---|---|---|
| [°2θ] | [cts] | [°2θ] | [Å] | [%] | [cts*°2θ] |
| 8.39 | 10953 | 0.134 | 10.539 | 87.0 | 1446 |
| 9.21 | 1626 | 0.117 | 9.600 | 12.9 | 188 |
| 9.86 | 12201 | 0.184 | 8.974 | 96.9 | 2214 |
| 11.47 | 12589 | 0.184 | 7.717 | 100.0 | 2285 |
| 13.95 | 752 | 0.151 | 6.347 | 6.0 | 112 |
| 14.73 | 1542 | 0.167 | 6.016 | 12.2 | 254 |
| 15.49 | 8430 | 0.184 | 5.721 | 67.0 | 1530 |
| 16.76 | 11218 | 0.184 | 5.289 | 89.1 | 2036 |
| 18.19 | 1999 | 0.151 | 4.877 | 15.9 | 2967 |
| 18.44 | 2642 | 0.134 | 4.812 | 21.0 | 349 |
| 18.57 | 1653 | 0.067 | 4.779 | 13.1 | 109 |
| 19.68 | 1688 | 0.117 | 4.511 | 13.4 | 195 |
| 20.20 | 1030 | 0.117 | 4.397 | 8.2 | 119 |
| 20.40 | 800 | 0.084 | 4.352 | 6.4 | 66 |
| 20.96 | 1979 | 0.134 | 4.239 | 15.7 | 261 |
| 21.23 | 1951 | 0.100 | 4.184 | 15.5 | 193 |
| 22.59 | 1574 | 0.117 | 3.936 | 12.5 | 182 |
| 22.72 | 1266 | 0.067 | 3.914 | 10.1 | 84 |
| 23.33 | 2466 | 0.117 | 3.813 | 19.6 | 285 |
| 23.55 | 4506 | 0.151 | 3.778 | 35.8 | 669 |
| 24.86 | 3635 | 0.167 | 3.581 | 28.9 | 600 |
| 25.50 | 4131 | 0.167 | 3.493 | 32.8 | 682 |
| 27.05 | 1128 | 0.184 | 3.297 | 9.0 | 205 |
| 27.75 | 1761 | 0.184 | 3.215 | 14.0 | 320 |
| 28.08 | 1609 | 0.201 | 3.178 | 12.8 | 319 |
| 29.08 | 1013 | 0.184 | 3.071 | 8.1 | 184 |
| 29.39 | 712 | 0.084 | 3.039 | 5.7 | 59 |
| 31.11 | 833 | 0.184 | 2.874 | 6.6 | 151 |
| 34.73 | 691 | 0.134 | 2.583 | 5.5 | 91 |
| 36.92 | 722 | 0.151 | 2.434 | 5.7 | 107 |

### Example 2

Preparation of Form B

Reverse Anti-Solvent Addition:

About 15 mg of sample material was dissolved in appropriate solvent to obtain a saturated solution. The saturated solution was added into a 20-mL glass vial with 5 mL of relative anti-solvent and the mixture was stirred at room temperature to induce crystallization. The crystals were agitated for 5 minutes and isolated and then analyzed with XRPD. The solvent pairs that gave rise to Form B are listed in Table 4 below. The diffractogram for Form B is shown in FIG. 2.

TABLE 4
Summary of experiments that generated Form B

| Solvent | Anti-solvent | Solid Form |
|---|---|---|
| NMP | water | Form B |
| DMSO | MeOH | Form B* |
| DMA | water | Form B |
| DMF | DCM | Form B |

*solution at RT was evaporated to induce crystallization

The XRPD peaks for Form B are listed in Table 5 below. The diffractogram for Form B is shown in FIG. 2.

TABLE 5
XRPD peaks of Form B

| Position | Heights | FWHM Left | d-spacing | Rel. Int. | Area |
|---|---|---|---|---|---|
| [°2θ] | [cts] | [°2θ] | [Å] | [%] | [cts*°2θ] |
| 7.17 | 6460 | 0.117 | 12.338 | 71.6 | 746 |
| 7.99 | 506 | 0.084 | 11.060 | 5.6 | 42 |
| 9.66 | 800 | 0.100 | 9.159 | 8.9 | 79 |
| 10.13 | 654 | 0.100 | 8.731 | 7.2 | 65 |
| 11.00 | 985 | 0.117 | 8.046 | 10.9 | 114 |
| 12.60 | 2663 | 0.167 | 7.024 | 29.5 | 439 |
| 12.95 | 1073 | 0.134 | 6.839 | 11.9 | 142 |
| 13.11 | 868 | 0.084 | 6.755 | 9.6 | 72 |
| 14.37 | 1423 | 0.134 | 6.165 | 15.8 | 188 |
| 14.54 | 556 | 0.050 | 6.093 | 6.2 | 28 |
| 14.81 | 1002 | 0.084 | 5.982 | 11.1 | 83 |
| 15.39 | 9028 | 0.100 | 5.758 | 100 | 894 |
| 16.07 | 869 | 0.100 | 5.514 | 9.6 | 86 |
| 16.62 | 5521 | 0.117 | 5.334 | 61.2 | 638 |
| 17.91 | 642 | 0.084 | 4.952 | 7.1 | 53 |
| 19.13 | 1347 | 0.167 | 4.640 | 14.9 | 222 |
| 20.81 | 3059 | 0.117 | 4.268 | 33.9 | 353 |
| 21.12 | 5869 | 0.117 | 4.206 | 65.0 | 678 |
| 21.42 | 1702 | 0.100 | 4.149 | 18.9 | 169 |
| 22.05 | 2229 | 0.134 | 4.032 | 24.7 | 294 |
| 22.34 | 5421 | 0.134 | 3.979 | 60.0 | 716 |
| 22.82 | 3498 | 0.151 | 3.896 | 38.7 | 519 |
| 23.22 | 1096 | 0.151 | 3.832 | 12.1 | 163 |
| 23.39 | 743 | 0.067 | 3.803 | 8.2 | 49 |
| 25.09 | 1218 | 0.084 | 3.549 | 13.5 | 101 |
| 25.40 | 1327 | 0.100 | 3.506 | 14.7 | 131 |
| 26.43 | 534 | 0.084 | 3.373 | 5.9 | 44 |
| 27.66 | 1365 | 0.167 | 3.225 | 15.1 | 225 |

### Example 3

Differential Scanning Calorimetry (DSC) Analysis

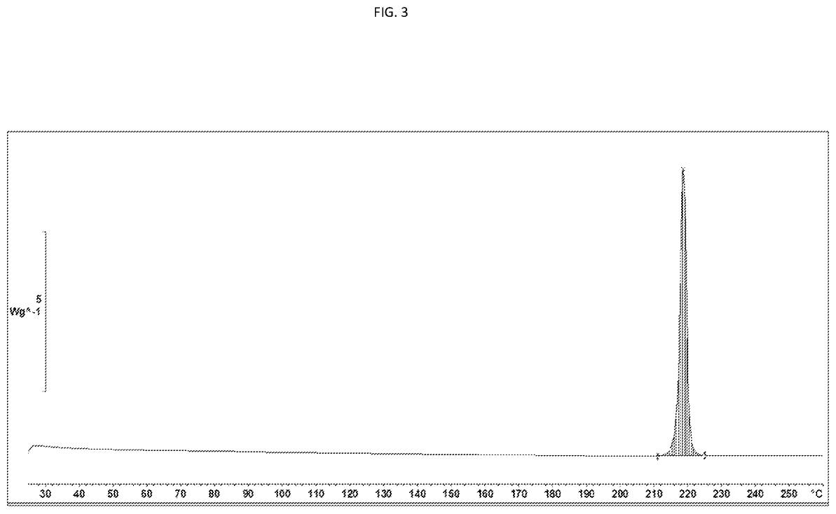

Form A did not show any significant events before melting at approximately 217° C. (onset 216.7° C.; endset 220.3° C.; peak 217.2° C.). The DSC thermogram is shown in FIG. 3.

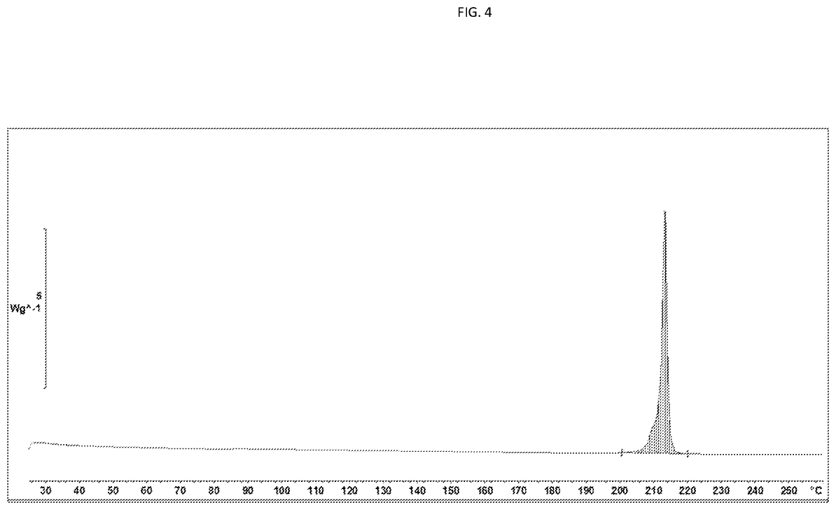

Form B did not show any significant events before melting at approximately 212° C. (onset 211.5° C.; endset 214.5° C.; peak 212.1° C.). The DSC thermogram is shown in FIG. 4.

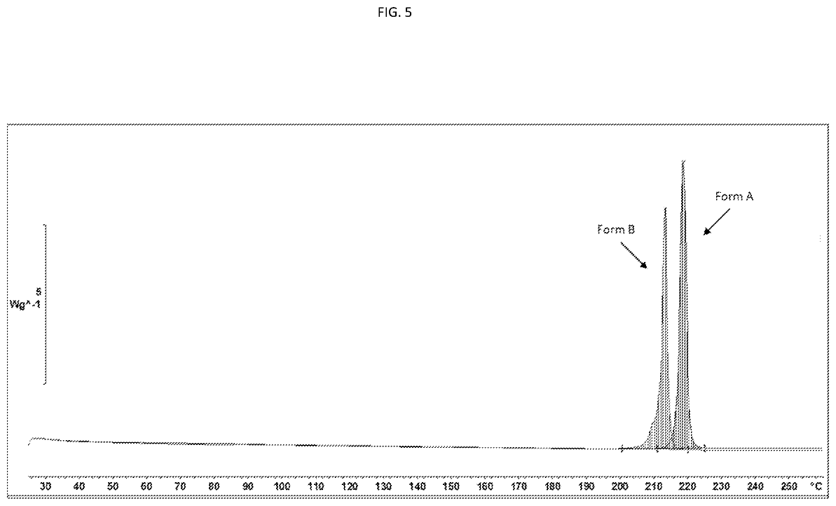

An overlay of the DSC thermograms of Forms A and B is shown in FIG. 5.

### Example 4

Thermogravimetric Analysis

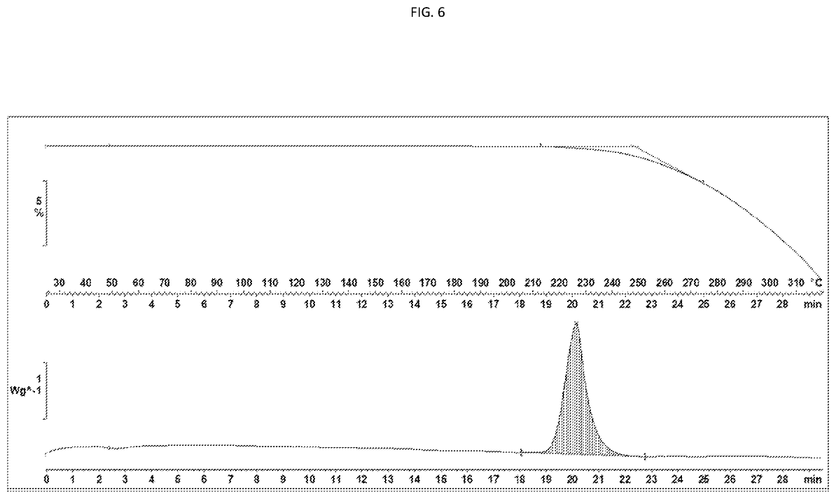

The sample of Form A did not show any weight loss before the melting of the sample at approximately 217° C. Degradation of the sample occurred at approximately 248° C. The TGA and the heatflow thermograms are shown in FIG. 6.

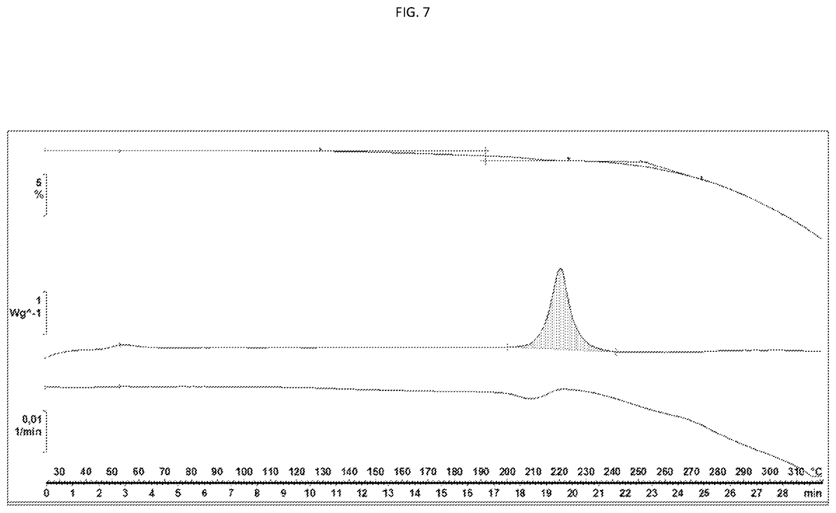

The sample of Form B showed a weight loss of approximately 1% in the range of 128-220° C., which was attributed to loss of isopropyl alcohol (determined by EGA analysis; not shown). Melting of the sample occurred at approximately 211° C., and degradation of the sample occurred at approximately 251° C. The TGA and the heatflow thermograms are shown in FIG. 7.

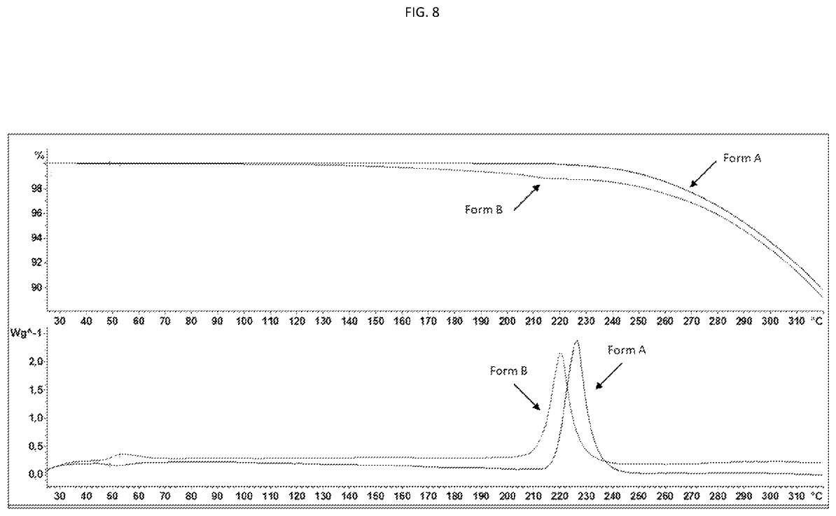

An overlay of the TGA and heatflow thermograms of Forms A and B is shown in FIG. 8.

### Example 5

Dynamic Vapour Sorption (DVS) Analysis

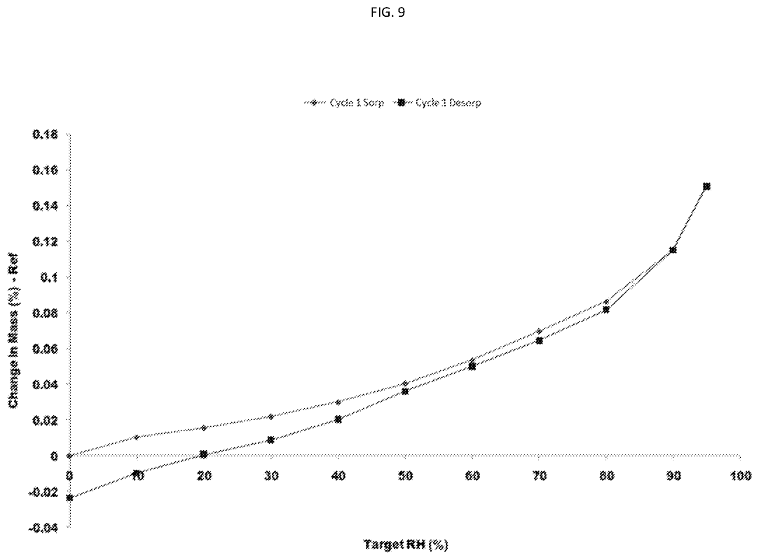

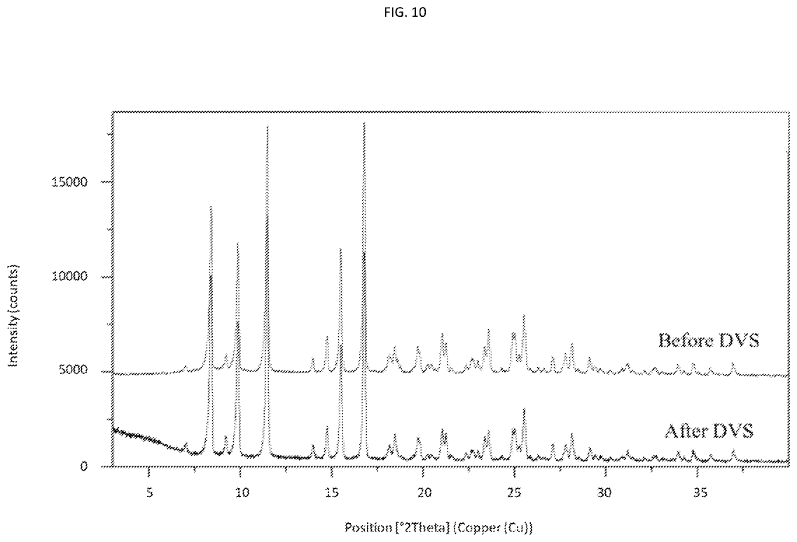

The hygroscopicity of Form A was investigated using DVS at 25° C. The isotherm plot is shown in FIG. 9. The water uptake at 25° C./80% RH was about 0.08%, which means that Form A is non-hygroscopic. A comparison of the X-ray powder diffractograms of the sample before and after the DVS analysis confirmed that no change of form occurred; see FIG. 10.

1. A crystalline anhydrate of X842.

2. The crystalline anhydrate of claim 1, wherein the anhydrate is stable at a relative humidity of 60% at a temperature of 25° C.

3. The crystalline anhydrate of claim 1 which is Form A, having an XRPD pattern, obtained with CuKα1-radiation, with at least peaks at °2θ values of 9.9±0.2 and 11.5±0.2.

4. The crystalline anhydrate of claim 1, wherein Form A has an XRPD pattern, obtained with CuKα1-radiation, with at least peaks at °2θ values of 9.9±0.2 and 11.5±0.2 and one or more of 8.4±0.2, 15.5±0.2 and 16.8±0.2.

5. The crystalline anhydrate of claim 4, wherein Form A has an XRPD pattern, obtained with CuKα1-radiation, with at least peaks at °2θ values of 8.4±0.2, 9.9±0.2, 11.5±0.2, 15.5±0.2 and 16.8±0.2.

6. The crystalline anhydrate of claim 4, wherein Form A has an XRPD pattern, obtained with CuKα1-radiation, with at least peaks at °2θ values of 8.4±0.2, 9.9±0.2, 11.5±0.2, 15.5±0.2, 16.8±0.2, 23.5±0.2, 24.9±0.2 and 25.5±0.2.

7. The crystalline anhydrate of claim 4, wherein Form A has an XRPD pattern, obtained with CuKα1-radiation, substantially as shown in FIG. 1.

8. The crystalline anhydrate of claim 1 which is Form B, having an XRPD pattern, obtained with CuKα1-radiation, with at least peaks at °2θ values of 7.2±0.2 and 15.4±0.2.

9. The crystalline anhydrate of claim 8, wherein Form B has an XRPD pattern, obtained with CuKα1-radiation, with at least peaks at °2θ values of 7.2±0.2 and 15.4±0.2, and one or more of 16.6±0.2, 21.1±0.2 and 22.3±0.2.

10. The crystalline anhydrate of claim 8, wherein Form B has an XRPD pattern, obtained with CuKα1-radiation, with at least peaks at °2θ values of 7.2±0.2, 15.4±0.2, 16.6±0.2, 21.1±0.2 and 22.3±0.2.

11. The crystalline anhydrate of claim 8, wherein Form B has an XRPD pattern, obtained with CuKα1-radiation, with at least peaks at °2θ values of 7.2±0.2, 12.6±0.2, 15.4±0.2, 16.6±0.2, 20.8±0.2, 21.1±0.2, 22.3±0.2 and 22.8±0.2.

12. The crystalline anhydrate of claim 8, wherein Form B has an XRPD pattern, obtained with CuKα1-radiation, substantially as shown in FIG. 2.

13. A composition comprising a crystalline anhydrate of X842, wherein the anhydrate is Form A, having an XRPD pattern, obtained with CuKα1-radiation, with at least peaks at °2θ values of 9.9±0.2 and 11.5±0.2.

14. The composition of claim 13, wherein the composition comprising Form A has a polymorphic purity of at least about 90%.

15. The composition of claim 13, wherein the composition comprising Form A contains less than about 15% by weight of Form B.

16. The composition of claim 13, wherein the composition comprising Form A is substantially free of Form B.

17. A composition comprising a crystalline anhydrate of X842, wherein the anhydrate is Form B, having an XRPD pattern, obtained with CuKα1-radiation, with at least peaks at °2θ values of 7.2±0.2 and 15.4±0.2.

18. The composition of claim 17, wherein the composition comprising Form B has a polymorphic purity of at least about 90%.

19. The composition of claim 17, wherein the composition comprising Form B contains less than about 15% by weight of Form A.

20. The composition of claim 17, wherein the composition comprising Form B is substantially free of Form A.

21. A pharmaceutical composition comprising a therapeutically effective amount of a crystalline anhydrate of X842, in association with one or more pharmaceutically acceptable excipients.

22. The pharmaceutical composition of claim 21, wherein the anhydrate is Form A, having an XRPD pattern, obtained with CuKα1-radiation, with at least peaks at⁰20 values of 9.9±0.2 and 11.5±0.2.

23. The pharmaceutical composition of claim 22, wherein Form A has a polymorphic purity of at least about 90%.

24. The pharmaceutical composition of claim 22, wherein Form A contains less than about 15% by weight of Form B.

25. The pharmaceutical composition of claim 22, wherein Form A is substantially free of Form B.

26. The pharmaceutical composition of claim 21, wherein a unit dose of the composition provides a Cₘᵢₙ of Linaprazan in a human of at least about 240 nmol/L after 22 hours following oral administration of the pharmaceutical composition to said human.
